(19) United States
(12) Patent Application Publication
(10) Pub. No.: US 2025/0009282 A1
(43) Pub. Date: Jan. 9, 2025

(54) AUTOMATIC DETECTION OF ANTERIOR COMMISSURE, POSTERIOR COMMISSURE, AND MID-SAGITTAL PLANE IN PATIENT SCANS

(71) Applicant: ClearPoint Neuro, Inc., Solana Beach, CA (US)

(72) Inventor: Lyubomir ZAGORCHEV, Burlington, MA (US)

(21) Appl. No.: 18/348,271

(22) Filed: Jul. 6, 2023

(51) Int. Cl. A61B 5/00; G06T 7/00; G16H 30/40

(52) U.S. Cl. CPC A61B 5/4064; G06T 7/0012; G16H 30/40; G06T 2207/30016

(57) ABSTRACT

Examples of the presently disclosed technology provide new systems and methods for automatically determining referential anatomical landmarks in patient scan coordinate spaces using a shape-constrained deformable brain model. The shape-constrained deformable brain model may comprise a computerized 3D representation of a non-patient-specific human brain that: (1) is symmetric about its mid-sagittal plane; and (2) preserves vertex-based correspondences-including mid-sagittal plane symmetry-during adaption to patient scans. Leveraging these unique features of the shape-constrained deformable brain model (i.e., mid-sagittal plane symmetry and preservation of vertex-based correspondences during adaption), examples can transform known/previously identified referential anatomical landmark coordinates in the shape-constrained deformable brain model coordinate space to a wide array of patient scan coordinate spaces (i.e., coordinate spaces for scans of a wide array of patients) in a highly accurate, efficient, and reproducible manner.

### TECHNICAL FIELD

The present disclosure relates generally to medical technologies, and more particularly, some examples relate to automatic detection of the anterior commissure, posterior commissure, and mid-sagittal plane in patient scans.

### BACKGROUND

Deep brain stimulation (DBS) involves placing leads/electrodes (i.e., DBS leads) within certain areas of a patient's brain. DBS leads produce electrical pulses that can regulate abnormal impulses, affect cells within the brain, etc. Clinically approved targets for DBS include the globus pallidus internus (GPi), the subthalamic nucleus (STN), the ventral intermedius nucleus (VIM), and the anterior nucleus of the thalamus (ANT).

### BRIEF DESCRIPTION OF THE DRAWINGS

The present disclosure, in accordance with one or more various examples, is described in detail with reference to the following figures. The figures are provided for purposes of illustration only and merely depict examples.

FIG. 1 depicts an example shape-constrained deformable brain model, in accordance with various examples of the presently disclosed technology.

FIG. 2 depicts a visual representation of the MNI152 template, in accordance with various examples of the presently disclosed technology

FIG. 3 depicts an example adaption of a shape-constrained deformable brain model to a scan of a patient's brain to generate a patient-specific 3D brain representation and a dense deformation field that transforms a mean shape of the shape-constrained deformable brain model to the scan of the patient's brain, in accordance with examples of the presently disclosed technology.

FIG. 4 depicts an example image of a human brain that identifies an anterior commissure (AC) and a posterior commissure (PC), in accordance with examples of the presently disclosed technology.

FIG. 5 depicts two example patient-specific 3D brain structure representations that may be used to determine mid-sagittal plane coordinates in a patient scan coordinate space, in accordance with examples of the presently disclosed technology.

FIG. 6 depicts an example flow diagram that may be used to determine AC coordinates, PC coordinates, and mid-sagittal plane coordinates in a patient scan coordinate space, in accordance with various examples of the presently disclosed technology.

FIG. 7 depicts another example flow diagram that may be used to determine AC coordinates, PC coordinates, and mid-sagittal plane coordinates in a patient scan coordinate space, in accordance with various examples of the presently disclosed technology.

FIG. 8 is an example computing component that may be used to implement various features of examples described in the present disclosure.

The figures are not exhaustive and do not limit the present disclosure to the precise form disclosed.

### DETAILED DESCRIPTION

Targeting brain structures for DBS procedures is typically done during pre-surgical planning. A common approach to targeting, sometimes referred to as “indirect targeting,” involves transforming one or more scans (e.g., MR scans, CT scans, PET scans, etc.) of a patient's brain to an atlas coordinate space (e.g., the Talaraich coordinate space). Consensus coordinates for DBS targets in the atlas coordinate space can then be used to estimate coordinates for DBS targets in a coordinate space of the patient scan(s) (i.e., the patient scan coordinate space). For example, if a patient scan is transformed to an atlas coordinate space, an inverse transformation will provide atlas/consensus DBS coordinates in the patient scan coordinate space. These coordinates can then be used for DBS lead implantation.

A patient scan can be transformed to an atlas coordinate space by identifying coordinates for referential anatomical landmarks in the patient scan coordinate space, and leveraging those referential anatomical landmark coordinates during transformation to the atlas coordinate space. Examples of referential anatomical landmarks for the brain include the anterior commissure (AC), the posterior commissure (PC), and the mid-sagittal plane.

Traditionally, surgeons would manually identify coordinates for these referential anatomical landmarks by reviewing patient scans. However, this manual identification process can be difficult, inefficient, and prone to human error. Accordingly, accurate, efficient, and reproducible techniques for automatically identifying/determining coordinates of referential anatomical landmarks in patient scan coordinate spaces can provide tremendous value to pre-surgical planning for DBS and other surgical interventions into the brain.

Against this backdrop, examples of the presently disclosed technology provide new systems and methods for automatically determining referential anatomical landmarks in patient scan coordinate spaces using a shape-constrained deformable brain model. The shape-constrained deformable brain model may comprise a computerized 3D representation of a non-patient-specific human brain that: (1) is symmetric about its mid-sagittal plane; and (2) preserves vertex-based correspondences-including mid-sagittal plane symmetry-during adaption to patient scans. Leveraging these unique features of the shape-constrained deformable brain model (i.e., mid-sagittal plane symmetry and preservation of vertex-based correspondences during adaption), examples can transform known/previously identified referential anatomical landmark coordinates in the shape-constrained deformable brain model coordinate space to a wide array of patient scan coordinate spaces (i.e., coordinate spaces for scans of a wide array of patients) in a highly accurate, efficient, and reproducible manner.

For instance, examples can adapt a shape-constrained deformable brain model to a scan of a patient's brain to generate a patient-specific 3D brain representation and a dense deformation field that transforms a mean shape of the shape-constrained deformable brain model to the scan of the patient's brain. Examples can then determine AC coordinates and PC coordinates in a coordinate space of the scan (i.e., the scan coordinate space) by using the generated dense deformation field to transform known/previously identified AC and PC coordinates in a coordinate space of the shape-constrained deformable brain model (i.e., the shape-constrained deformable brain model coordinate space) to the scan coordinate space. In some cases, the shape-constrained deformable brain model coordinate space may be defined by the MNI152 template (i.e., the shape-constrained deformable brain model coordinate space may comprise the MNI152 coordinate space). As alluded to above, the same known/previously identified AC and PC coordinates in the shape-constrained deformable brain model coordinate space can be transformed into a wide array of patient scan coordinate spaces in a highly accurate and reproducible manner.

Leveraging the mid-sagittal plane symmetry of the shape-constrained deformable brain model which is preserved during adaption to the scan, examples can determine mid-sagittal plane coordinates in the scan coordinate space by: (1) determining a pair of vertices of the patient-specific 3D brain representation that are symmetric with respect to each other about a mid-sagittal plane of the patient-specific 3D brain representation; and (2) determining the mid-sagittal plane coordinates in the scan coordinate space by computing a midpoint of a parametric line connecting the pair of vertices. In some cases, the pair of vertices may comprise corresponding left and right vertices of a mid-sagittal plane-symmetric patient-specific 3D brain structure representation of the patient-specific 3D brain representation. In other cases, the pair of vertices may comprise corresponding vertices of a left patient-specific 3D brain structure representation and a right patient-specific 3D brain structure of the patient-specific 3D brain representation that are symmetric with respect to each other about the mid-sagittal plane of the patient-specific 3D brain representation.

In some implementations, examples can determine mid-sagittal plane coordinates in the scan coordinate space with increased accuracy by determining multiple pairs of mid-sagittal plane-symmetric vertices and performing least squares regression to fit a plane corresponding to the mid-sagittal plane in the scan coordinate space. For instance, examples can: (1) determine a first pair of vertices of the patient-specific 3D brain representation that are symmetric with respect to each other about a mid-sagittal plane of the patient-specific 3D brain representation; (2) determine a second pair of vertices of the patient-specific 3D brain representation that are symmetric with respect to each other about the mid-sagittal plane of the patient-specific 3D brain representation; (3) compute a mid-point of a first parametric line connecting the first pair of vertices and a mid-point of a second parametric line connecting the second pair of vertices; and (4) determine mid-sagittal plane coordinates in the scan coordinate space by performing a least squares regression on the computed mid-points.

In certain implementations, examples can transform the scan of the patient's brain to an atlas coordinate space (e.g., the Talaraich coordinate space) using the AC coordinates, PC coordinates, and mid-sagittal plane coordinates determined in the scan coordinate space. As alluded to above, transforming a patient scan to an atlas coordinate space such as the Talaraich coordinate space can be an important step in pre-surgical planning for certain procedures. For example, consensus coordinates for DBS targets in the atlas coordinate space can be used to estimate coordinates for DBS targets in the scan coordinate space. Namely, if the scan of the patient's brain is transformed to the atlas coordinate space, an inverse transformation will provide atlas/consensus DBS coordinates in the scan coordinate space. These coordinates can then be used for DBS lead implantation. Accordingly, in some implementations, examples can determine DBS lead implantation coordinates in the scan coordinate space based on the above-described transformation of the scan of the patient's brain to the atlas coordinate space.

In other implementations, examples can provide the AC, PC, and mid-sagittal plane coordinates determined in the scan coordinate space to a user (e.g., a surgeon) and/or an automated (i.e., computerized) surgical planning system. The user and/or automated surgical planning system can then leverage the AC, PC, and mid-sagittal plane coordinates determined in the scan coordinate space to transform the scan to an atlas coordinate space (e.g., the Talaraich coordinate space) for the purposes of planning a surgical intervention into the patient's brain (e.g., a DBS procedure).

Examples of the present technology provide numerous benefits. Determining coordinates for referential anatomical landmarks in a patient scan coordinate space, and leveraging the determined referential anatomical landmark coordinates in the patient scan coordinate space to transform a patient scan to an atlas coordinate space—can be critical steps in pre-surgical planning for DBS and other surgical interventions into the brain. As alluded to above, traditionally, surgeons would manually identify coordinates for these referential anatomical landmarks by reviewing patient scans. However, this manual identification process can be difficult, inefficient, and prone to human error. Accordingly, the present technology's accurate, efficient, and reproducible methodology for automatically determining coordinates of referential anatomical landmarks in patient scan coordinate spaces provides tremendous value to pre-surgical planning for DBS and other surgical interventions into the brain.

Examples of the presently disclosed technology will be described in greater detail in conjunction with the following FIGS.

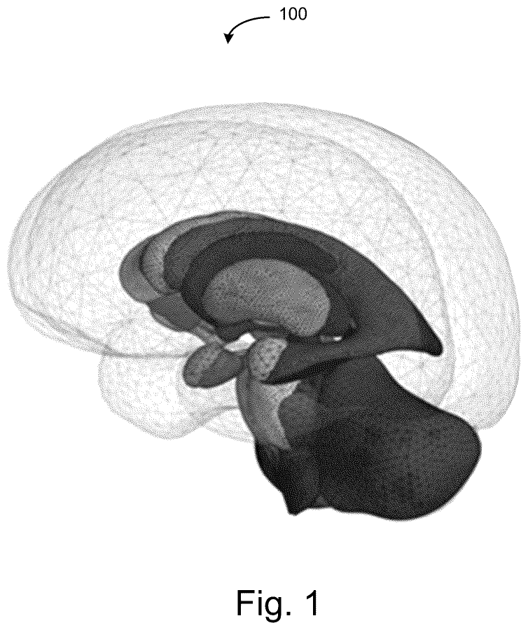

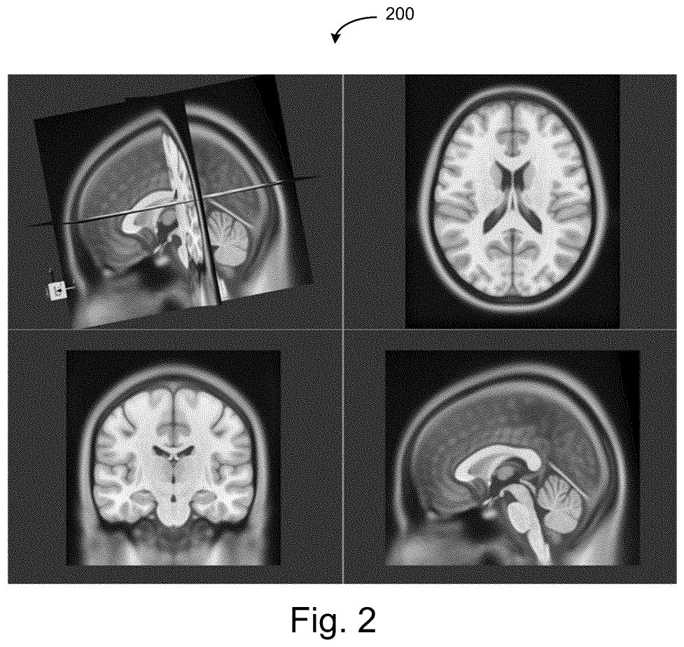

FIG. 1 depicts an example shape-constrained deformable brain model 100, in accordance with various examples of the presently disclosed technology. Shape-constrained deformable brain model 100 may be a computerized 3D representation of a generalized human brain (i.e., a non-patient-specific 3D representation of the human brain) that: (1) is symmetric about its mid-sagittal plane; and (2) preserves vertex-based correspondences-including mid-sagittal plane symmetry-during adaption to patient scans using shape-constrained deformation. Shape-constrained deformable brain model 100 can be derived as an average/mean representation from a set of training data. As described in conjunction with FIG. 2, in various implementations the average/mean representation of shape-constrained deformable brain model 100 may be defined by the MNI152 template. Accordingly, the coordinate space of shape-constrained deformable brain model 100 may comprise the MNI152 coordinate space.

As depicted, shape-constrained deformable brain model 100 may comprise mesh elements and mesh vertices at the junctions of adjoining/adjacent mesh elements. Each mesh element of shape-constrained deformable brain model 100 may represent a different brain region. In the specific example of FIG. 1, the mesh elements of shape-constrained deformable brain model 100 comprise triangles, but in other examples mesh elements may comprise different shapes.

In general, a mesh may refer to a representation of a larger domain (e.g., a volume or surface) comprised of smaller discrete cells called mesh elements, and mesh vertices at the junctions of adjacent/adjoining mesh elements. Meshes can be used to compute solutions to equations across individual mesh elements, which then can be used to approximate solutions over the larger domain.

As depicted (and as will be discussed below), shape-constrained deformable brain model 100 may comprise individual 3D segments/sub-representations representing various structures of the brain (e.g., the cerebral cortex, sub-cortical structures, etc.).

While in the specific example of FIG. 1 shape-constrained deformable brain model 100 comprises a 3D mesh, in other examples shape-constrained deformable brain model 100 may comprise other types of representations that can define geometry of anatomical brain regions (e.g., a parametric surface, a point cloud, etc.).

FIG. 2 depicts a visual representation 200 of the MNI152 template, in accordance with various examples of the presently disclosed technology. The MNI152 template is an atlas created by registration and averaging of MR data from 152 patients/subjects aligned in the same coordinate space (i.e., the MNI152 coordinate space). As alluded to above, in various implementations the average/mean shape of shape-constrained deformable brain model 100 may be defined by the MNI152 template. Accordingly, the coordinate space of shape-constrained deformable brain model 100 may comprise the MNI152 coordinate space.

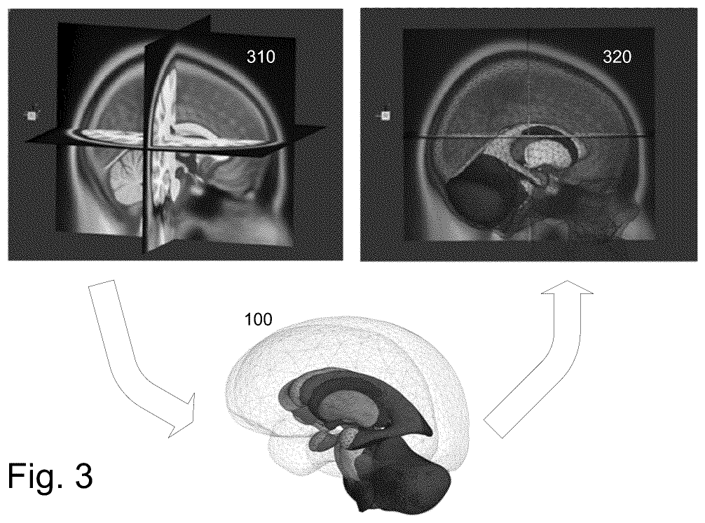

FIG. 3 depicts an example adaption of shape-constrained deformable brain model 100 to a scan 310 of a patient's brain to generate a patient-specific 3D brain representation 320 and a dense deformation field that transforms a mean shape of shape-constrained deformable brain model 100 to the scan of the patient's brain, in accordance with examples of the presently disclosed technology. As depicted in FIG. 3, patient-specific 3D brain representation 320 may comprise individual 3D sub-representations/segments representing individual brain structures of the patient (i.e., patient-specific 3D brain structure representations). The patient-specific 3D brain structure representations may represent the various brain structures (e.g., sub-cortical structures) of the patient's brain.

As alluded to above, shape-constrained deformable brain model 100 may be a computerized 3D representation of a generalized human brain (i.e., a non-patient-specific 3D representation of the human brain) that: (1) is symmetric about its mid-sagittal plane; and (2) preserves vertex-based correspondences-including mid-sagittal plane symmetry-during adaption to patient scans using shape-constrained deformation. Namely, the process of adaption generates a dense deformation field that transforms voxels from the coordinate space of shape-constrained deformable brain model 100 (i.e., the shape-constrained deformable brain model 100 coordinate space) to the coordinate space of scan 310 (i.e., the scan 310 coordinate space). The dense deformation field can leverage shape-constrained deformation to constrain deformation to an apriori derived mean shape (i.e., shape-constrained deformable brain model 100). The dense deformation field can use a penalty term estimated from the mean shape (i.e., estimated from shape-constrained deformable brain model 100) that prevents topological changes during adaptation, which may be an iterative process. Segmentation (i.e., generation of individual/segmented patient-specific 3D brain structure representations) may gradually deform the mean shape (i.e., gradually deform shape-constrained deformable brain model 100) to match the patient-specific scan (i.e., scan 310). In other words, shape may be constrained to the mean shape (i.e., constrained to shape-constrained deformable brain model 100), which can grow or shrink without morphing into a different shape.

Through the above-described adaptation, correspondences can be preserved between vertices of shape-constrained deformable brain model 100 and vertices of patient-specific 3D brain representation 320. Accordingly, vertices representing the AC and PC in shape-constrained deformable brain model 100 will correspond with vertices representing the AC and PC in patient-specific 3D brain representation 320. Leveraging this unique feature adaption/transformation feature (i.e., preservation of vertex-based correspondences), examples can transform known/previously identified AC coordinates and PC coordinates in the shape-constrained deformable brain model 100 coordinate space to a wide array of patient coordinate spaces (including the scan 310 coordinate space) in a highly accurate, efficient, and reproducible manner.

As described above, the mid-sagittal plane symmetry of shape-constrained deformable brain model 100 is also preserved during adaption to scan 310. Accordingly, like shape-constrained deformable brain model 100, patient-specific 3D brain representation 320 may also be symmetric about its respective mid-sagittal plane. As alluded to above (and as will be described in greater detail below), examples can leverage these unique features to determine mid-sagittal plane coordinates in the scan 310 coordinate space by: (1) determining a pair of vertices of patient-specific 3D brain representation 320 that are symmetric with respect to each other about a mid-sagittal plane of patient-specific 3D brain representation 320; and (2) determining the mid-sagittal plane coordinates in the scan 310 coordinate space by computing a midpoint of a parametric line connecting the pair of vertices.

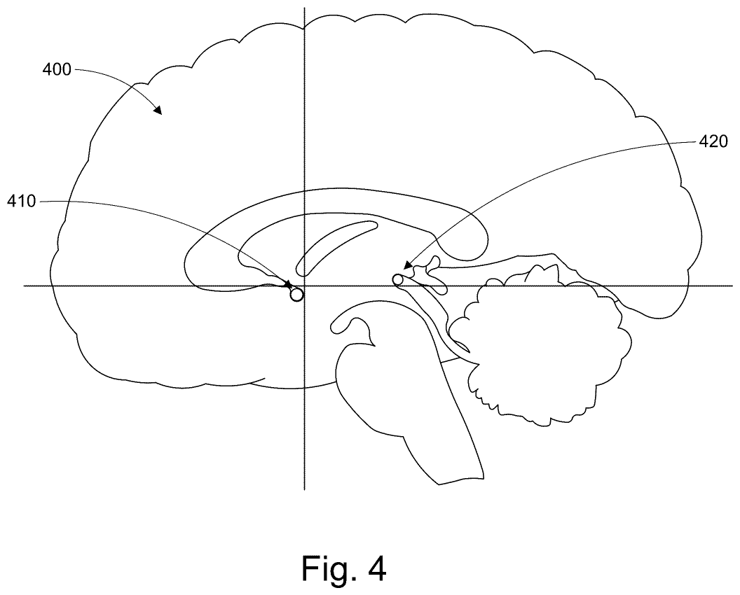

FIG. 4 depicts an example image of a human brain 400 that identifies an anterior commissure (AC) 410 and a posterior commissure (PC) 420, in accordance with examples of the presently disclosed technology.

As described above, a patient scan can be transformed to an atlas coordinate space by identifying coordinates for referential anatomical landmarks in the patient scan coordinate space, and leveraging those referential anatomical landmark coordinates during transformation to the atlas coordinate space. Examples of referential anatomical landmarks for the brain include the anterior commissure (AC), the posterior commissure (PC), and the mid-sagittal plane.

Traditionally, surgeons would manually identify coordinates for these referential anatomical landmarks by reviewing patient scans. However, this manual identification process can be difficult, inefficient, and prone to human error. Accordingly, the present technology's accurate, efficient, and reproducible methodology for automatically determining coordinates of referential anatomical landmarks in patient scan coordinate spaces provides tremendous value to pre-surgical planning for DBS and other surgical interventions into the brain.

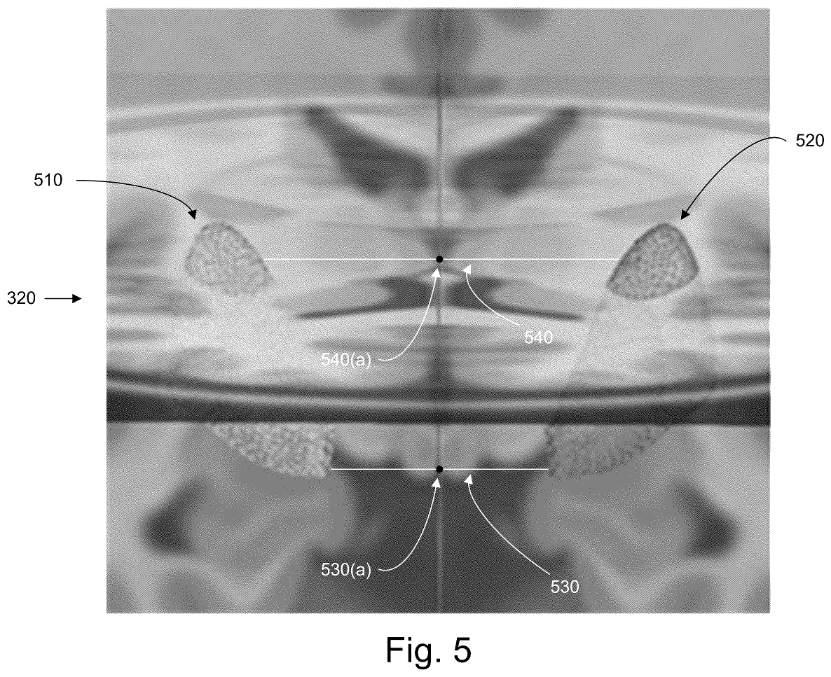

FIG. 5 depicts example patient-specific 3D brain structure representations 510 and 520 that may be used to determine mid-sagittal plane coordinates in a patient scan coordinate space, in accordance with examples of the presently disclosed technology. Here patient-specific 3D brain structure representations 510 and 520 may be individual segments/sub-representations of patient-specific 3D brain representation 320. As depicted, patient-specific 3D brain structure representations 510 and 520 represent a patient's left and right putamen respectively. The left and right putamen are symmetric with respect to each other about the mid-sagittal plane.

As alluded to above, mid-sagittal plane coordinates in a patient scan coordinate space (e.g., the scan 310 coordinate space) can be determined using the inherent symmetry of the shape constrained deformable brain model (e.g., shape constrained deformable brain model 100) used to generate patient-specific 3D brain representation 320. In other words, leveraging the mid-sagittal plane symmetry of the shape-constrained deformable brain model which is preserved during adaption to patient scans, examples can determine mid-sagittal plane coordinates in a patient scan coordinate space by: (1) determining a pair of vertices of patient-specific 3D brain representation 320 that are symmetric with respect to each other about a mid-sagittal plane of patient-specific 3D brain representation 320; and (2) determining the mid-sagittal plane coordinates in the scan coordinate space by computing a midpoint of a parametric line connecting the pair of vertices. In some cases, the pair of vertices may comprise corresponding left and right vertices of a mid-sagittal plane-symmetric patient-specific 3D brain structure representation of patient-specific 3D brain representation 320 (i.e., a patient-specific 3D brain structure representation which is symmetric about the mid-sagittal plane of patient-specific 3D brain representation 320). In other cases, the pair of vertices may comprise corresponding vertices of a left patient-specific 3D brain structure representation and a right patient-specific 3D brain structure of patient-specific 3D brain representation 320 that are symmetric with respect to each other about the mid-sagittal plane of patient-specific 3D brain representation 320.

FIG. 5 specifically illustrates the latter case. Namely, patient-specific 3D brain structure representations 510 and 520 may respectively comprise left and right patient-specific 3D brain structure representations of patient-specific 3D brain representation 320 that are symmetric with respect to each other (i.e., mirror images of each other) about the mid-sagittal plane of patient-specific 3D brain representation 320. Accordingly, if the shape (e.g., a triangular mesh) of patient-specific 3D brain structure representation 510 (representing the patient's left putamen) has vertices 1 . . . . N, then the shape of patient-specific 3D brain structure representation 520 (representing the patient's right putamen) will have the same 1 . . . . N vertices on the opposite side of the mid-sagittal plane. Again, this unique feature results from the inherent symmetry of the shape constrained deformable brain model (e.g., shape constrained deformable brain model 100) used to generate patient-specific 3D brain structure representations 510 and 520. Consequently, examples can determine mid-sagittal plane coordinates in the scan coordinate space by computing a midpoint 530 (a) of a parametric line 530 connecting corresponding vertices of patient-specific 3D brain structure representations 510 and 520 respectively. More formally, the parametric line can be written as:

$$p = {p}_{left} + t ( {p}_{right} - {p}_{left} )$$

where p stands for point coordinates (X,Y,Z) on the left and right side of the mid-sagittal plane and t is a parameter along the line. Coordinates that belong to the mid-sagittal plane can be obtained at t=0.5.

As alluded to above, in certain implementations, examples can determine mid-sagittal plane coordinates in the scan coordinate space with increased accuracy by determining multiple pairs of mid-sagittal plane-symmetric vertices and performing least squares regression to fit a plane corresponding to the mid-sagittal plane in the scan coordinate space. For instance, examples can: (1) determine a first pair of vertices of patient-specific 3D brain representation 320 that are symmetric with respect to each other about a mid-sagittal plane of patient-specific 3D brain representation 320 (e.g., a first pair of corresponding vertices of patient-specific 3D brain structure representations 510 and 520 respectively); (2) determine a second pair of vertices of patient-specific 3D brain representation 320 that are symmetric with respect to each other about the mid-sagittal plane of patient-specific 3D brain representation 320 (e.g., a second pair of corresponding vertices of patient-specific 3D brain structure representations 510 and 520 respectively); (3) compute a mid-point (e.g., mid-point 530 (a)) of a first parametric line (e.g., parametric line 530) connecting the first pair of vertices and a mid-point (e.g., mid-point 540 (a)) of a second parametric line (e.g., parametric line 540) connecting the second pair of vertices; and (4) determine mid-sagittal plane coordinates in the scan coordinate space by performing a least squares regression on the computed mid-points.

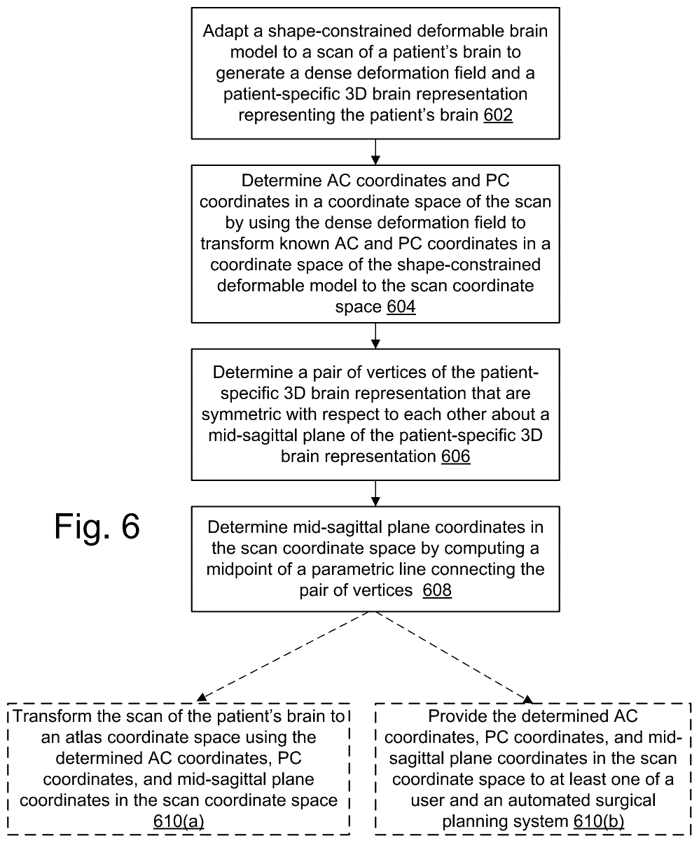

FIG. 6 depicts an example flow diagram that may be used to determine AC coordinates, PC coordinates, and mid-sagittal plane coordinates in a patient scan coordinate space, in accordance with various examples of the presently disclosed technology.

At operation 602, examples can adapt a shape-constrained deformable brain model to a scan of a patient's brain to generate a dense deformation field and a patient-specific 3D brain representation representing the patient's brain. The dense deformation field can transform a mean shape of the shape-constrained deformable brain model to the scan of the patient's brain. As alluded to above, the shape-constrained deformable brain model may comprise a computerized 3D representation of a non-patient-specific human brain that: (1) is symmetric about its mid-sagittal plane; and (2) preserves vertex-based correspondences-including mid-sagittal plane symmetry-during adaption to patient scans. Leveraging these unique features of the shape-constrained deformable brain model (i.e., mid-sagittal plane symmetry and preservation of vertex-based correspondences during adaption), examples can transform known/previously identified referential anatomical landmark coordinates in the shape-constrained deformable brain model coordinate space to a wide array of patient scan coordinate spaces (i.e., coordinate spaces for scans of a wide array of patients) in a highly accurate, efficient, and reproducible manner. As alluded to above, in some cases, the shape-constrained deformable brain model coordinate space may be defined by the MNI152 template (i.e., the shape-constrained deformable brain model coordinate space may comprise the MNI152 coordinate space).

At operation 604, examples can determine anterior commissure (AC) coordinates and posterior commissure (PC) coordinates in a coordinate space of the scan by using the dense deformation field to transform known/previously identified AC and PC coordinates in a coordinate space of the shape-constrained deformable model to the scan coordinate space. As alluded to above, the same known/previously identified AC and PC coordinates in the shape-constrained deformable brain model coordinate space can be transformed into a wide array of patient scan coordinate spaces in a highly accurate and reproducible manner.

Leveraging the mid-sagittal plane symmetry of the shape-constrained deformable brain model which is preserved during adaption to the scan, at operation 606, examples can determine a pair of vertices of the patient-specific 3D brain representation that are symmetric with respect to each other about a mid-sagittal plane of the patient-specific 3D brain representation. Relatedly, at operation 608, examples can determine the mid-sagittal plane coordinates in the scan coordinate space by computing a midpoint of a parametric line connecting the pair of vertices. In some cases, the pair of vertices may comprise corresponding left and right vertices of a mid-sagittal plane-symmetric patient-specific 3D brain structure representation of the patient-specific 3D brain representation. In other cases, the pair of vertices may comprise corresponding vertices of a left patient-specific 3D brain structure representation and a right patient-specific 3D brain structure of the patient-specific 3D brain representation that are symmetric with respect to each other about the mid-sagittal plane of the patient-specific 3D brain representation.

In certain implementations, at operation 610 (a), examples can transform the scan of the patient's brain to an atlas coordinate space (e.g., the Talaraich coordinate space) using the AC coordinates, PC coordinates, and mid-sagittal plane coordinates determined in the scan coordinate space. As alluded to above, transforming a patient scan to an atlas coordinate space such as the Talaraich coordinate space can be an important step in pre-surgical planning for certain procedures. For example, consensus coordinates for DBS targets in the atlas coordinate space can be used to estimate coordinates for DBS targets in the scan coordinate space. Namely, if the scan is transformed to the atlas coordinate space, an inverse transformation will provide atlas/consensus DBS coordinates in the scan coordinate space. These coordinates can then be used for DBS lead implantation. Accordingly, in some implementations examples can determine DBS lead implantation coordinates in the scan coordinate space based on the above-described transformation of the scan of the patient's brain to the atlas coordinate space.

In other implementations, at operation 610 (b), examples can provide the AC, PC, and mid-sagittal plane coordinates determined in the scan coordinate space to a user (e.g., a surgeon) and/or an automated (i.e., computerized) surgical planning system. The user and/or automated surgical planning system can then leverage the AC, PC, and mid-sagittal plane coordinates determined in the scan coordinate space to transform the scan to an atlas coordinate space (e.g., the Talaraich coordinate space) for the purposes of planning a surgical intervention into the patient's brain (e.g., a DBS procedure).

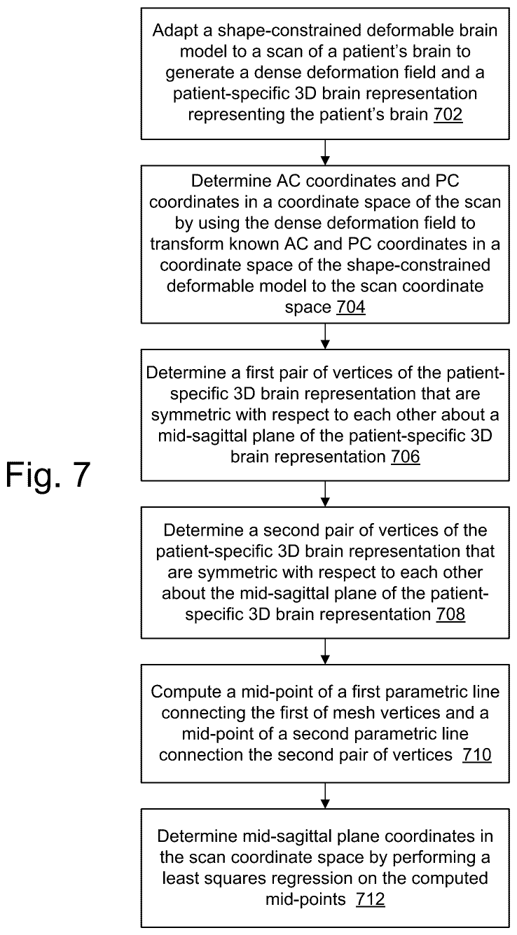

FIG. 7 depicts another example flow diagram that may be used to determine AC coordinates, PC coordinates, and mid-sagittal plane coordinates in a patient scan coordinate space, in accordance with various examples of the presently disclosed technology.

At operation 702, examples can adapt a shape-constrained deformable brain model to a scan of a patient's brain to generate a dense deformation field and a patient-specific 3D brain representation representing the patient's brain. This operation may be performed in the same/similar manner as described in conjunction with operation 602.

At operation 704, examples can determine anterior commissure (AC) coordinates and posterior commissure (PC) coordinates in a coordinate space of the scan by using the dense deformation field to transform known/previously identified AC and PC coordinates in a coordinate space of the shape-constrained deformable model to the scan coordinate space. This operation may be performed in the same/similar manner as described in conjunction with operation 604.

As alluded to above, in some implementations, examples can determine mid-sagittal plane coordinates in the scan coordinate space with increased accuracy by determining multiple pairs of mid-sagittal plane-symmetric vertices and performing least squares regression to fit a plane corresponding to the mid-sagittal plane in the scan coordinate space.

For instance, at operation 706, examples can determine a first pair of vertices of the patient-specific 3D brain representation that are symmetric with respect to each other about a mid-sagittal plane of the patient-specific 3D brain representation. Relatedly, at operation 708, examples can determine a second pair of vertices of the patient-specific 3D brain representation that are symmetric with respect to each other about the mid-sagittal plane of the patient-specific 3D brain representation. At operation 710, examples can compute a mid-point of a first parametric line connecting the first pair of vertices and a mid-point of a second parametric line connecting the second pair of vertices. Accordingly, at operation 712, examples can determine mid-sagittal plane coordinates in the scan coordinate space by performing a least squares regression on the computed mid-points.

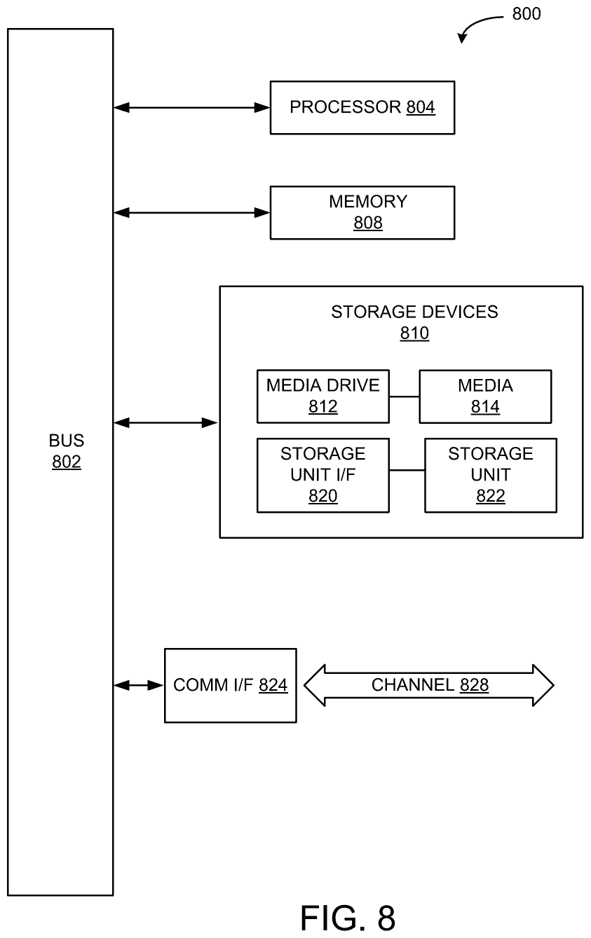

As used herein, the terms circuit and component might describe a given unit of functionality that can be performed in accordance with one or more examples of the present application. As used herein, a component might be implemented utilizing any form of hardware, software, or a combination thereof. For example, one or more processors, controllers, ASICs, PLAS, PALs, CPLDs, FPGAs, logical components, software routines or other mechanisms might be implemented to make up a component. Various components described herein may be implemented as discrete components or described functions and features can be shared in part or in total among one or more components. In other words, as would be apparent to one of ordinary skill in the art after reading this description, the various features and functionality described herein may be implemented in any given application. They can be implemented in one or more separate or shared components in various combinations and permutations. Although various features or functional elements may be individually described or claimed as separate components, it should be understood that these features/functionality can be shared among one or more common software and hardware elements. Such a description shall not require or imply that separate hardware or software components are used to implement such features or functionality.

Where components are implemented in whole or in part using software, these software elements can be implemented to operate with a computing or processing component capable of carrying out the functionality described with respect thereto. One such example computing component is shown in FIG. 8. Various examples are described in terms of this example-computing component 800. For example, computing component 800 may be used to implement the operations described in conjunction with FIGS. 6-7. After reading this description, it will become apparent to a person skilled in the relevant art how to implement the application using other computing components or architectures.

Referring now to FIG. 8, computing component 800 may represent, for example, computing or processing capabilities found within a self-adjusting display, desktop, laptop, notebook, and tablet computers. They may be found in hand-held computing devices (tablets, PDA's, smart phones, cell phones, palmtops, etc.). They may be found in workstations or other devices with displays, servers, or any other type of special-purpose or general-purpose computing devices as may be desirable or appropriate for a given application or environment. Computing component 800 might also represent computing capabilities embedded within or otherwise available to a given device. For example, a computing component might be found in other electronic devices such as, for example, portable computing devices, and other electronic devices that might include some form of processing capability.

Computing component 800 might include, for example, one or more processors, controllers, control components, or other processing devices. Processor 804 might be implemented using a general-purpose or special-purpose processing engine such as, for example, a microprocessor, controller, or other control logic. Processor 804 may be connected to a bus 802. However, any communication medium can be used to facilitate interaction with other components of computing component 800 or to communicate externally.

Computing component 800 might also include one or more memory components, simply referred to herein as main memory 808. For example, random access memory (RAM) or other dynamic memory, might be used for storing information and instructions to be executed by processor 804. Main memory 808 might also be used for storing temporary variables or other intermediate information during execution of instructions to be executed by processor 804. Computing component 800 might likewise include a read only memory (“ROM”) or other static storage device coupled to bus 802 for storing static information and instructions for processor 804.

The computing component 800 might also include one or more various forms of information storage mechanism 810, which might include, for example, a media drive 812 and a storage unit interface 820. The media drive 812 might include a drive or other mechanism to support fixed or removable storage media 814. For example, a hard disk drive, a solid-state drive, a magnetic tape drive, an optical drive, a compact disc (CD) or digital video disc (DVD) drive (R or RW), or other removable or fixed media drive might be provided. Storage media 814 might include, for example, a hard disk, an integrated circuit assembly, magnetic tape, cartridge, optical disk, a CD or DVD. Storage media 814 may be any other fixed or removable medium that is read by, written to or accessed by media drive 812. As these examples illustrate, the storage media 814 can include a computer usable storage medium having stored therein computer software or data.

In alternative examples, information storage mechanism 810 might include other similar instrumentalities for allowing computer programs or other instructions or data to be loaded into computing component 800. Such instrumentalities might include, for example, a fixed or removable storage unit 822 and an interface 820. Examples of such storage units 822 and interfaces 820 can include a program cartridge and cartridge interface, a removable memory (for example, a flash memory or other removable memory component) and memory slot. Other examples may include a PCMCIA slot and card, and other fixed or removable storage units 822 and interfaces 820 that allow software and data to be transferred from storage unit 822 to computing component 800.

Computing component 800 might also include a communications interface 824. Communications interface 824 might be used to allow software and data to be transferred between computing component 800 and external devices. Examples of communications interface 824 might include a modem or softmodem, a network interface (such as Ethernet, network interface card, IEEE 802.XX or other interface). Other examples include a communications port (such as for example, a USB port, IR port, RS232 port Bluetooth® interface, or other port), or other communications interface. Software/data transferred via communications interface 824 may be carried on signals, which can be electronic, electromagnetic (which includes optical) or other signals capable of being exchanged by a given communications interface 824. These signals might be provided to communications interface 824 via a channel 828. Channel 828 might carry signals and might be implemented using a wired or wireless communication medium. Some examples of a channel might include a phone line, a cellular link, an RF link, an optical link, a network interface, a local or wide area network, and other wired or wireless communications channels.

In this document, the terms “computer program medium” and “computer usable medium” are used to generally refer to transitory or non-transitory media. Such media may be, e.g., memory 808, storage unit 820, media 814, and channel 828. These and other various forms of computer program media or computer usable media may be involved in carrying one or more sequences of one or more instructions to a processing device for execution. Such instructions embodied on the medium, are generally referred to as “computer program code” or a “computer program product” (which may be grouped in the form of computer programs or other groupings). When executed, such instructions might enable the computing component 800 to perform features or functions of the present application as discussed herein.

It should be understood that the various features, aspects and functionality described in one or more of the individual examples are not limited in their applicability to the particular example with which they are described. Instead, they can be applied, alone or in various combinations, to one or more other examples, whether or not such examples are described and whether or not such features are presented as being a part of a described example. Thus, the breadth and scope of the present application should not be limited by any of the above-described exemplary examples.

Terms and phrases used in this document, and variations thereof, unless otherwise expressly stated, should be construed as open ended as opposed to limiting. As examples of the foregoing, the term “including” should be read as meaning “including, without limitation” or the like. The term “example” is used to provide exemplary instances of the item in discussion, not an exhaustive or limiting list thereof. The terms “a” or “an” should be read as meaning “at least one,” “one or more” or the like; and adjectives such as “conventional,” “traditional,” “normal,” “standard,” “known.” Terms of similar meaning should not be construed as limiting the item described to a given time period or to an item available as of a given time. Instead, they should be read to encompass conventional, traditional, normal, or standard technologies that may be available or known now or at any time in the future. Where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “component” does not imply that the aspects or functionality described or claimed as part of the component are all configured in a common package. Indeed, any or all of the various aspects of a component, whether control logic or other components, can be combined in a single package or separately maintained and can further be distributed in multiple groupings or packages or across multiple locations.

Additionally, the various examples set forth herein are described in terms of exemplary block diagrams, flow charts and other illustrations. As will become apparent to one of ordinary skill in the art after reading this document, the illustrated examples and their various alternatives can be implemented without confinement to the illustrated examples. For example, block diagrams and their accompanying description should not be construed as mandating a particular architecture or configuration.

1. A non-transitory computer-readable storage medium including instructions that, when executed by at least one processor of a computing system, cause the computing system to perform a method comprising:
adapting a shape-constrained deformable brain model to a scan of a patient's brain to generate a dense deformation field and a patient-specific 3D brain representation;
determining anterior commissure (AC) coordinates and posterior commissure (PC) coordinates in a coordinate space of the scan by using the dense deformation field to transform known AC and PC coordinates in a coordinate space of the shape-constrained deformable model to the scan coordinate space;
determining a pair of vertices of the patient-specific 3D brain representation that are symmetric with respect to each other about a mid-sagittal plane of the patient-specific 3D brain representation; and
determining mid-sagittal plane coordinates in the scan coordinate space by computing a midpoint of a parametric line connecting the pair of vertices.

2. The non-transitory computer-readable storage medium of claim 1, wherein:
the shape-constrained deformable brain model is symmetric about a mid-sagittal plane of the shape-constrained deformable brain model; and
the mid-sagittal plane symmetry of the shape-constrained deformable brain model is preserved during adaption to the scan of the patient's brain.

3. The non-transitory computer-readable storage medium of claim 2, wherein the pair of vertices comprise corresponding left and right vertices of a mid-sagittal plane-symmetric patient-specific 3D brain structure representation of the patient-specific 3D brain representation.

4. The non-transitory computer-readable storage medium of claim 2, wherein the pair of vertices comprise corresponding vertices of a left patient-specific 3D brain structure representation and a right patient-specific 3D brain structure of the patient-specific 3D brain representation that are symmetric with respect to each other about the mid-sagittal plane of the patient-specific 3D brain representation.

5. The non-transitory computer-readable storage medium of claim 1, wherein the method further comprises:
providing the determined AC coordinates, PC coordinates, and mid-sagittal plane coordinates in the scan coordinate space to at least one of a user and an automated surgical planning system.

6. The non-transitory computer-readable storage medium of claim 1, wherein the method further comprises:
transforming the scan of the patient's brain to an atlas coordinate space using the determined AC coordinates, PC coordinates, and mid-sagittal plane coordinates in the scan coordinate space.

7. The non-transitory computer-readable storage medium of claim 6, wherein the method further comprises:
determining deep brain stimulation (DBS) lead implantation coordinates in the scan coordinate space based on the transformation of the scan to the atlas coordinate space.

8. The non-transitory computer-readable storage medium of claim 6, wherein the atlas coordinate space comprises a Talaraich coordinate space.

9. The non-transitory computer-readable storage medium of claim 1, wherein the shape-constrained deformable brain model coordinate space comprises an MNI152 coordinate space.

10. A method, comprising:
adapting a shape-constrained deformable brain model to a scan of a patient's brain to generate a dense deformation field and a patient-specific 3D brain representation;
determining AC coordinates and PC coordinates in a coordinate space of the scan by using the dense deformation field to transform known AC and PC coordinates in a coordinate space of the shape-constrained deformable model to the scan coordinate space;
determining a first pair of vertices of the patient-specific 3D brain representation that are symmetric with respect to each other about a mid-sagittal plane of the patient-specific 3D brain representation;
determining a second pair of vertices of the patient-specific 3D brain representation that are symmetric with respect to each other about the mid-sagittal plane of the patient-specific 3D brain representation;
computing a mid-point of a first parametric line connecting the first pair of vertices and a mid-point of a second parametric line connection the second pair of vertices; and
determining mid-sagittal plane coordinates in the scan coordinate space by performing a least squares regression on the computed mid-points.

11. The method of claim 10, wherein:
the shape-constrained deformable brain model is symmetric about a mid-sagittal plane of the shape-constrained deformable brain model; and
the mid-sagittal plane symmetry of the shape-constrained deformable brain model is preserved during adaption to the scan of the patient's brain.

12. The method of claim 10, further comprising:
providing the determined AC coordinates, PC coordinates, and mid-sagittal plane coordinates in the scan coordinate space to at least one of a user and an automated surgical planning system.

13. The method of claim 12, further comprising:
transforming the scan of the patient's brain to an atlas coordinate space using the determined AC coordinates, PC coordinates, and mid-sagittal plane coordinates in the scan coordinate space.

14. The method of claim 13, further comprising:
determining DBS lead implantation coordinates in the scan coordinate space based on the transformation of the scan of the patient's brain to the atlas coordinate space.

15. The method of claim 13, wherein the atlas coordinate space comprises a Talaraich coordinate space.

16. The method of claim 10, wherein the shape-constrained deformable brain model coordinate space comprises an MNI152 coordinate space.

17. A system comprising:
one or more processing resources; and
a non-transitory computer-readable medium, coupled to the one or more processing resources, having stored therein instructions that when executed by the one or more processing resources cause the system to perform a method comprising:
adapting a shape-constrained deformable brain model to a scan of a patient's brain to generate a dense deformation field and a patient-specific 3D brain representation, wherein the shape-constrained deformable brain model is symmetric about a mid-sagittal plane of the shape-constrained deformable brain model and the mid-sagittal plane symmetry of the shape-constrained deformable brain model is preserved during adaption to the scan of the patient's brain;
determining AC coordinates and PC coordinates in a coordinate space of the scan by using the dense deformation field to transform known AC and PC coordinates in a coordinate space of the shape-constrained deformable model to the scan coordinate space;
determining a pair of vertices of the patient-specific 3D brain representation that are symmetric with respect to each other about a mid-sagittal plane of the patient-specific 3D brain representation; and
determining mid-sagittal plane coordinates in the scan coordinate space by computing a midpoint of a parametric line connecting the pair of vertices.

18. The system of claim 17, wherein the shape-constrained deformable brain model comprises a computerized 3D representation of a non-patient-specific human brain that preserves vertex-based correspondences during adaption to patient scans.

19. The system of claim 17, wherein the pair of vertices comprise at least one of:
corresponding left and right vertices of a mid-sagittal plane-symmetric patient-specific 3D brain structure representation of the patient-specific 3D brain representation; and
corresponding vertices of a left patient-specific 3D brain structure representation and a right patient-specific 3D brain structure of the patient-specific 3D brain representation that are symmetric with respect to each other about the mid-sagittal plane of the patient-specific 3D brain representation.

20. The system of claim 17, wherein the shape-constrained deformable brain model coordinate space comprises an MNI152 coordinate space.
